(19) United States
(12) Patent Application Publication
(10) Pub. No.: US 2024/0294455 A1
(43) Pub. Date: Sep. 5, 2024

(54) SYNTHESIS OF VINYLATED HYDROXY ESTERS WITH APPLICABILITY IN SIGNAL ENHANCED MAGNETIC RESONANCE IMAGING

(71) Applicant: MAX-PLANCK-GESELLSCHAFT ZUR FÖRDERUNG DER WISSENSCHAFTEN E. V., München (DE)

(72) Inventors: Stefan GLÖGGLER, Göttingen (DE); Sergey KORCHAK, Göttingen (DE); Anil P. JATAP, Göttingen (DE); Philip SAUL, Göttingen (DE); Denis MOLL, Göttingen (DE)

(73) Assignee: MAX-PLANCK-GESELLSCHAFT ZUR FÖRDERUNG DER WISSENSCHAFTEN E. V., München (DE)

(21) Appl. No.: 18/571,275

(22) PCT Filed: Jun. 16, 2022
(86) PCT No.: PCT/EP2022/066535

§ 371 Date: Dec. 18, 2023

(30) Foreign Application Priority Data
Jun. 18, 2021 (EP) 21180465.3

(51) Int. Cl. C07C 67/31; C07B 59/00; C07C 67/307; C07C 67/317; C07C 69/63; C07C 69/732; C07C 201/12; C07C 205/34; C07C 227/18; C07C 229/08

(52) U.S. Cl. CPC C07C 67/31; C07B 59/001; C07C 67/307; C07C 67/317; C07C 69/63; C07C 69/732; C07C 201/12; C07C 205/34; C07C 227/18; C07C 229/08; C07B 2200/05

(57) ABSTRACT

The present invention relates to a method for preparing a compound suitable for signal enhanced magnetic resonance imaging comprising the steps of vinylating a mono-, di- or tricarboxylic acid comprising a moiety -Q-Z with Q being O or N and Z being a protecting group by using vinyl acetate, cleaving Z, and if Z has not already been converted into an alcohol upon cleavage, converting Q into an alcohol either by using nitrite or by converting Q into bromine followed by hydrolysis yielding a vinyl hydroxy ester. The compounds used may be partly or fully deuterated. Furthermore, the present invention relates to a vinyl hydroxy ester as well as an intermediate of its synthesis, wherein the vinyl moiety of the vinyl hydroxy ester and of the intermediate is partly or fully deuterated.

The present invention relates to methods to synthesize vinylated hydroxy esters that are suitable for the preparation of hyperpolarized agents that enhance NMR signals.

Furthermore, the present invention relates to vinylated hydroxy esters per se as well as intermediates of their synthesis.

### BACKGROUND OF THE INVENTION

The phenomenon of nuclear magnetic resonance (NMR) and its tomography modality magnetic resonance imaging (MRI) has wide applicability in analytics and clinical diagnostics. NMR is an intrinsically insensitive phenomenon which is why hyperpolarization strategies were devised to improve the sensitivity. Hyperpolarization is thereby a process that enhances NMR signals by several orders of magnitude compared to the normal/thermally polarized signal. In the past years the use of hyperpolarized metabolites was introduced to the field of preclinical and clinical research to study diseases even in patients. The state-of-the-art technique is dynamic nuclear polarization (DNP). For this procedure typically ¹³C and ¹⁵N isotopically enriched molecules are used whereby the most prominent example is ¹³C enriched pyruvate. The molecules are cooled down to cryogenic temperatures at and below 2 K in the presence of radicals inside a dedicated super-conducting high-field magnet. At that low temperature the irradiation with microwaves over tens of minutes to hours leads to the polarization transfer of the highly polarized electron spins of the radicals to the heteronucleus of the desired molecule. Heteronuclei are spins other than protons, ¹H. Protons can also be polarized via the described procedures but are less relevant for preclinical or clinical studies. Subsequently, the hyperpolarized molecules of interest are rapidly thawed and can be used as signal enhanced magnetic resonance contrast agents as they allow to probe metabolic conversion directly in real-time.

Another technique of hyperpolarization is the para-hydrogen induced polarization (PHIP). It is a faster approach than DNP and polarizes metabolites in seconds rather than tens of minutes to hours. In order to signal enhance metabolites suitable precursor molecules are required that can
A) be rapidly reacted with the para-spin isomer of hydrogen,
B) yield high degrees of signal enhancements and
C) can be quickly converted into the molecule of interest.

Using the PHIP approach several metabolites including acetate, lactate and pyruvate where hyperpolarized. However, the obtained polarization of isotopically enriched compounds stays one order of magnitude behind that achievable via DNP. If similar polarization degrees are achieved via PHIP for such metabolic contrast agents this opens up the opportunity to make the production of contrast agents cost-efficient, orders of magnitude faster and widely applicable to health-care institutions because the PHIP technique does not require a dedicated high-field magnet. In contrast, only portable low-field devices are required in which para-hydrogen is reacted via suitable precursors.

So far, NMR experiments have been devised to theoretically deliver optimal results for producing signal-enhanced contrast agents. However, the chemical precursors that promote maximal signal enhancements for important metabolites including lactate and pyruvate do not exist. Literature studies have shown that vinyl carboxylic acids (e.g. vinyl acetate, vinyl lactate and vinyl pyruvate) are the most promising precursor molecules. Although the molecules are known, the isotopic labelling with deuterium has not been achieved so far. To achieve optimal signal enhancements via PHIP not only a ¹³C atom needs to be included into the precursor but ideally at least the vinyl functionality should be deuterated. This is a challenge that has so far not been overcome.

The present invention relates to new chemical procedures that allow to synthesize vinyl esters of hydroxy esters. The most prominent representative of this class of molecules is vinyl lacate (α-hydroxy ester). Other molecules of immediate interest are vinyl esters of hydroxybutyrate, malate and citrate (hydroxy esters). Although the metabolite's free acid is often more desirable the uncleaved ester can also be used as contrast agents. In contrast to the known methods and products described above, the present invention allows cost-effective preparation of vinylated hydroxy esters that are suitable for the preparation of hyperpolarized agents.

Based on the above-mentioned state of the art, the objective of the present invention is to provide means and methods to synthesize vinylated hydroxy esters that are suitable for the preparation of hyperpolarized agents that enhance NMR signals. This objective is attained by the subject-matter of the independent claims of the present specification, with further advantageous embodiments described in the dependent claims, examples, FIGURES and general description of this specification.

### SUMMARY OF THE INVENTION

A first aspect of the invention relates to a method for preparing a compound suitable for signal enhanced magnetic resonance imaging. The method comprises the steps of
a) vinylation of a compound of formula (VIIa), (VIIb) or (VIIc), particularly (VIIa),

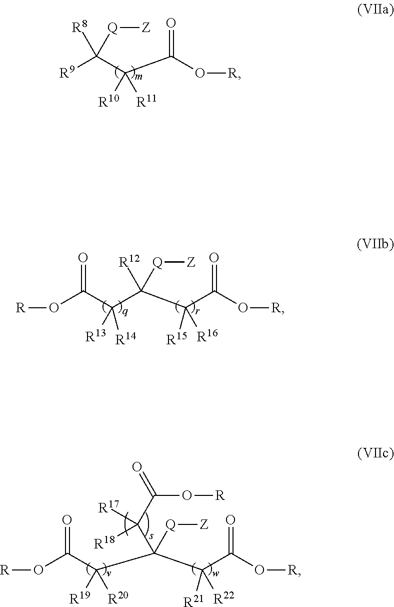

by using vinyl acetate of formula (II),

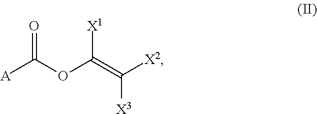

yielding an intermediate of formula (VIIIa), (VIIIb) or (VIIIc), particularly (VIIIa),

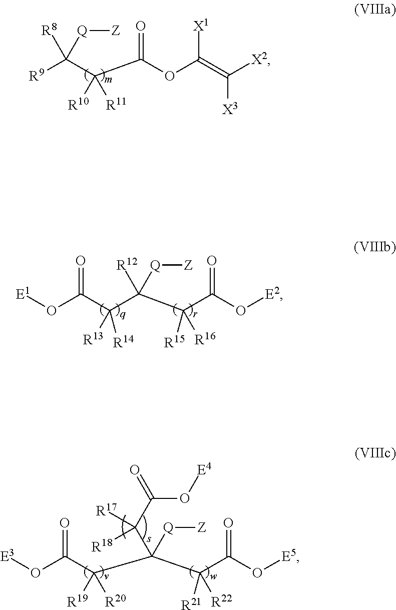

b) cleaving Z by using UV light or by using a suitable agent, particularly TFA,
c) in case of using TFA in step (b), TFA is removed,
d) converting Q into an alcohol in the presence of nitrite, or
   converting Q into bromine in the presence of nitrite followed by hydrolysis
   yielding a vinyl hydroxy ester of formula (IXa), (IXb) or (IXc), particularly (IXa),

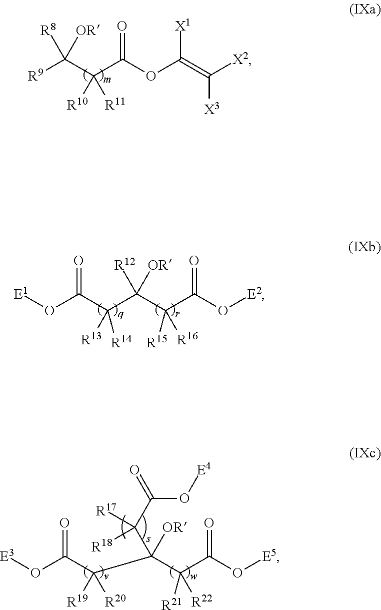

wherein
each R is independently of any other R selected from H and D,
R′ is selected from H and D, particularly H,
R⁸, R⁹ and R¹² are independently of each other selected from H, D, and a fully or partly deuterated alkyl,
each R¹⁰, R¹³, R¹⁵, R¹⁷, R¹⁸ and R²¹ is independently of any other R¹⁰, R¹³, R¹⁵, R¹⁷, R¹⁸ or R²¹ selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
Q is O or N,
Z is a protecting group,
X¹, X² and X³ are independently of each other H or D,
A is —CH₃, —CH₂D, —CHD₂ or —CD₃,
E¹, E², E³, E⁴ and E⁵ are independently of each other H, D or wherein

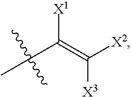

X¹, X² and X³ are as defined above and wherein at least one moiety selected from E¹ and E² and at least one moiety selected from E³, E⁴ and E⁵ is

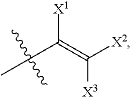

and m, q, r, s, v and w are independently of each other integers between 0 and 16.

A second aspect of the invention relates to a vinyl hydroxy ester of formula (IXa), (IXb) or (IXc), particularly (IXa),

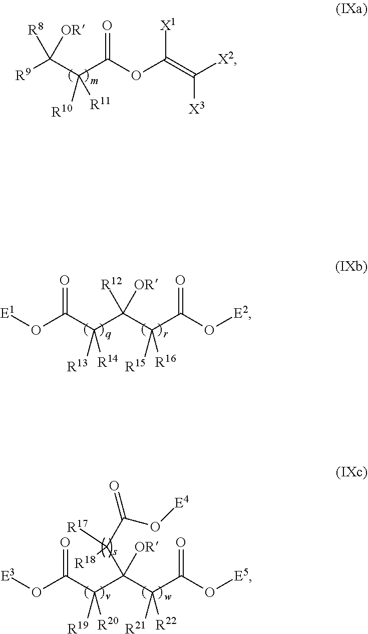

wherein
each R′ is independently of any other R′ selected from H and D,
R⁸, R⁹ and R¹² are independently of each other selected from H, D, and a fully or partly deuterated alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
X¹, X² and X³ are independently of each other H or D, wherein at least one X¹, X² and X³ is D,
A is —CH₃, —CH₂D, —CHD₂ or —CD₃, and
E¹, E², E³, E⁴ and E⁵ are independently of each other H, D or

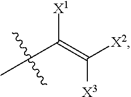

wherein X¹, X² and X³ are as defined above and wherein at least one moiety selected from E¹ and E² and at least one moiety selected from E³, E⁴ and E⁵ is

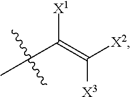

m, q, r, s, v and w are independently of each other integers between 0 and 16.

A third aspect of the invention relates to an intermediate of formula (VIIIa), (VIIIb) or (VIIIc), particularly (VIIIa),

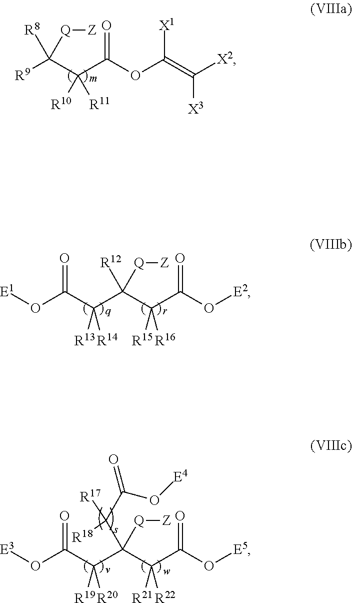

wherein
R⁸, R⁹ and R¹² are independently of each other selected from H, D, and a fully or partly deuterated alkyl,
each R¹⁰, R¹³, R¹⁵, R¹⁷, R¹⁹ and R²¹ is independently of any other R¹⁰, R¹³, R¹⁵, R¹⁷, R¹⁸ or R²¹ selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
Q is O or N,
Z is a protecting group,
X¹, X² and X³ are independently of each other H or D, wherein at least one X¹, X² and X³ is D,
A is —CH₃, —CH₂D, —CHD₂ or —CD₃, and
E¹, E², E³, E⁴ and E⁵ are independently of each other H, D or

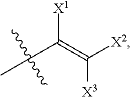

wherein X¹, X² and X³ are as defined above and wherein at least one moiety selected from E¹ and E² and at least one moiety selected from E³, E⁴ and E⁵ is

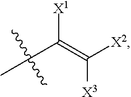

m, q, r, s, v and w are independently of each other integers between 0 and 16.

### DESCRIPTION

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

Reference to heteroatoms in the formula encompass, unless defined otherwise, all suitable forms depending on the neighbouring definitions (e.g. —N═ or —NH—). The suitable form is readily understood by one of ordinary skill in the art. For example in case of a definition -Q-Z with Q being N being and Z being an amine protection group is clear that depending on the chosen protecting group N could encompass —N═ or —NH—, e.g. in case of Z being Boc Q is —NH—.”

As used herein, including in the appended claims, the singular forms “a,” “or,” and “the” include plural referents unless the context clearly dictates otherwise.

The term “alkyl” in the context of the present specification relates to a saturated linear or branched hydrocarbon. For example, a C₁-C₆ alkyl in the context of the present specification relates to a saturated linear or branched hydrocarbon having 1, 2, 3, 4, 5 or 6 carbon atoms.

Non-limiting examples for a C₁-C₆ alkyl include methyl, ethyl, propyl, 1-methylethyl (isopropyl), n-butyl, 2-methylpropyl, tert-butyl, n-pentyl, 2-methylbutyl, 3-methylbutyl, 1,1-dimethylpropyl, 1,2-dimethylpropyl, n-hexyl, 3-methyl-2-pentyl, and 4-methyl-2-pentyl. Similarly, the term C₁₋₁₆-alkyl relates to a saturated linear or branched hydrocarbon having 1 to 16 carbon atoms.

The term “hydroxybutyric acid” relates to 2-hydroxybutanoic acid as well as to 3-hydroxybutanoic acid.

The term “hydroxybutyrate” relates to alpha-hydroxybutyrate (α-hydroxybutyrate) as well as to 3-hydroxybutyrate (β-hydroxybutyrate).

Unless defined otherwise, all technical and scientific terms used herein have the same meaning as commonly understood by one of ordinary skill in the art.

### DETAILED DESCRIPTION

A first aspect of the invention relates to a method for preparing a compound suitable for signal enhanced magnetic resonance imaging. The method comprises the steps of
a) vinylation of a compound of formula (VIIa), (VIIb) or (VIIc), particularly (VIIa),

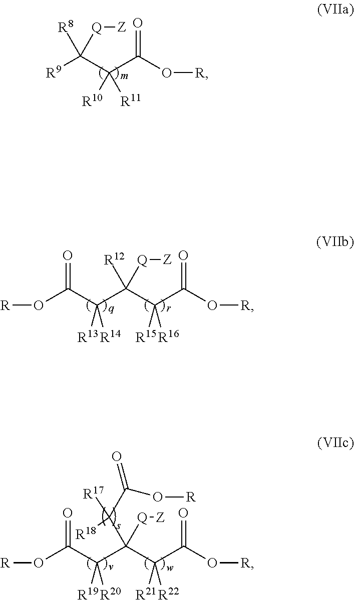

by using vinyl acetate of formula (II),

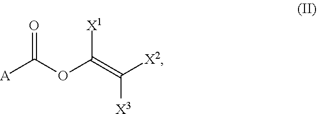

yielding an intermediate of formula (VIIIa), (VIIIb) or (VIIIc), particularly (VIIIa),

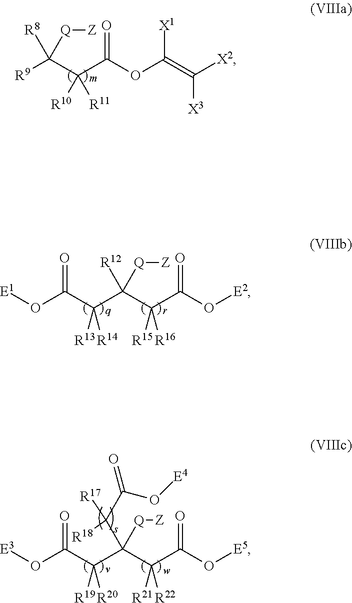

b) cleaving Z by using UV light or by using a suitable agent, particularly TFA,
c) in case of using TFA in step (b), TFA is removed,
d) converting Q into an alcohol in the presence of nitrite, particularly NaNO₂ or tert-butyl-NO₂, or
   converting Q into bromine in the presence of nitrite, particularly NaNO₂ or tert-butyl-NO₂, more particularly NaNO₂, followed by hydrolysis
   yielding a vinyl hydroxy ester of formula (IXa), (IXb) or (IXc), particularly (IXa),

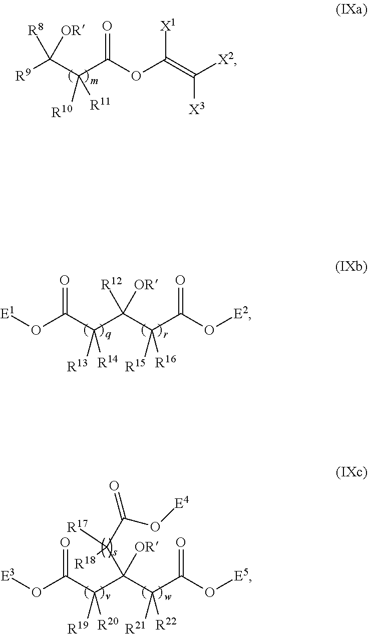

wherein
each R is independently of any other R selected from H and D, particularly H,
R′ is selected from H and D, particularly H,
R⁸, R⁹ and R¹² are independently of each other selected from H, D, and a fully or partly deuterated alkyl, particularly C₁₋₁₆-alkyl, more particularly C₁₋₆-alkyl, even more particularly C₁₋₃-alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group, particularly selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
Q is O or N,
Z is a protecting group, particularly selected from an amine protection group and a photolabile protection group, more particularly selected from, Boc, Fmoc, benzyl
carbamate, acetamide, trifluoroacetamide, and

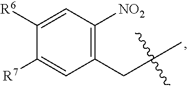

wherein
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
   R^{a} being H or a C₁₋₃-alkyl,
   p being an integer between 0 and 6,
X¹, X² and X³ are independently of each other H or D, particularly D,
A is —CH₃, —CH₂D, —CHD₂ or —CD₃, particularly —CD₃,
E¹, E², E³, E⁴ and E⁵ are independently of each other H, D or

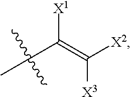

wherein X¹, X² and X³ are as defined above and wherein at least one moiety selected from E¹ and E² and at least one moiety selected from E³, E⁴ and E⁵ is

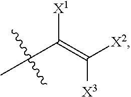

and
m, q, r, s, v and w are independently of each other integers between 0 and 16, particularly between 0 and 6, more particularly between 0 and 3.

The method described in the fourth aspect of the invention aims to provide a vinyl hydroxy ester that is suitable for the use in signal enhanced magnetic resonance imaging. The vinyl hydroxy ester is a precursor that promotes maximal signal enhancement for metabolites such as lactate. To achieve optimal signal enhancement via PHIP not only a ¹³C spins needs to be included into the precursor but ideally at least the vinyl functionality should be deuterated.

Known attempts to produce suitable vinyl hydroxy esters suffer from low yields (ca. 10%) and loss of deuterium.

The method of the fourth aspect of the invention makes use of a protection strategy that allows vinylation and deprotection under mild conditions without loss of deuterium.

A protected amino acid or protected hydroxy carboxylic acid is vinylated in a first step.

Removal of the protecting group in case of having started with a protected hydroxy carboxylic acid yields a vinyl hydroxy ester. For example, a protecting group such as nitrobenzoyl may be removed by applying UV light.

In case of having started with a protected amino acid, the amine may be deprotected and subsequently converted to an alcohol using nitrite. When the deprotection has been performed by using TFA, TFA has to be removed prior to hydrolysis.

Alternatively, an additional bromination step in the presence of nitrite is performed before hydrolysis at basic pH. Also here, TFA has to be removed prior to hydrolysis. Very good yields are achieved (>80%).

For the preparation of a contrast agent for signal enhanced magnetic resonance imaging, a carbon of the vinyl hydroxy ester is hyperpolarized through 1H-polarization transfer via pH₂.

The ester obtained may be used as such as contrast agent or may be cleaved by hydrolysis to use the hyperpolarized metabolite such as lactate as contrast agent.

In certain embodiments, m is 0 or 1.

In certain embodiments, R⁸ is selected from H, D, —CH₃, —CH₂D, —CHD₂ and —CD₃.

In certain embodiments, R⁹, R¹¹ are independently of each other H or D.

In certain embodiments, R¹⁰ is selected from H, D, NH₂, —CH₃, —CH₂D, —CHD₂ and —CD₃, particularly from H, D and NH₂.

In certain embodiments, each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group, particularly the NH₂ moiety is protected by a protecting group in case of Q being N or NH.

In certain embodiments, each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety is protected by a protecting group.

Suitable protecting groups for the NH₂ moiety are known to a person of skill in the art. The protecting group for the NH₂ moiety at any of the positions R¹⁰, R¹³, R¹⁵, R¹⁷, R¹⁹ or R²¹ is selected from suitable protecting groups that are orthogonal to the protecting group Z of the moiety Q. In this context, “orthogonal” relates to a protection strategy which allows the specific deprotection of one protective group, e.g. removal of the protecting group Z, without affecting other protecting groups, e.g. the protecting group for the NH₂ moiety at any of the positions R¹⁰, R¹³, R¹⁵, R¹⁷, R¹⁹ or R²¹. For example, a skilled person might use Fmoc as protecting group at any of the positions R¹⁰, R¹³, R¹⁵, R¹⁷, R¹⁹ or R²¹ if Z is Boc. After removal of the Boc protecting group by using a suitable reagent such as TFA, the Fmoc protecting group might be removed by using piperidine in a suitable solvent such as DMF (dimethylformamide). An alternative orthogonal protection strategy might be achieved if Fmoc or Boc is replaced by a photolabile protecting group.

In certain embodiments, the protecting group for the NH₂ moiety at any of the positions R¹⁰, R¹³, R¹⁵, R¹⁷, R¹⁹ or R²¹ may be selected from Boc, Fmoc, benzyl carbamate, acetamide, trifluoroacetamide and a photolabile protecting group, particularly selected from Boc, Fmoc and a photolabile protecting group, wherein said protecting group is selected in such a way that said protecting group is orthogonal to the protecting group Z of the moiety Q.

In certain embodiment, the photolabile protecting group is a nitrobenzyl-based protecting group, particularly selected from

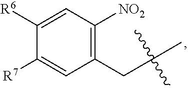

wherein
R⁶ or R⁷ is H or —OCH₃, and the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with R^{a} being H or a C₁₋₃-alkyl, p being an integer between 0 and 6.

In certain embodiments, the protecting group for the NH₂ moiety may be selected from Boc, Fmoc, benzyl carbamate, acetamide and trifluoroacetamide, particularly selected from Boc and Fmoc, wherein said protecting group is chosen in such a way that said protection group is orthogonal to the protecting group Z of the moiety Q.

In certain embodiments, R⁸ is selected from H, D, —CH₃, —CH₂D, —CHD₂ and —CD₃, R⁹, R¹¹ are independently of each other H or D, R¹⁰ is selected from H, D, NH₂, —CH₃, —CH₂D, —CHD₂ and —CD₃, particularly from H, D and NH₂, more particularly from H and D.

In certain embodiments, q is 0 or 1 and r is 0 or 1.

In certain embodiments, q is 0 and r is 1.

In certain embodiments, R¹², R¹³, R¹⁴, R¹⁵, R¹⁶ are independently of each other H or D.

In certain embodiments, s is 0 or 1, v is 0 or 1, w is 0 or 1.

In certain embodiments, s is 0.

In certain embodiments, s is 0 and v is 1 and w is 1.

In certain embodiments, R¹⁷, R¹⁸, R¹⁹, R²⁰, R²¹, R²² are independently of each other H or D.

In certain embodiments, R⁸ is selected from H, D, —CH₃, —CH₂D, —CHD₂ and —CD₃, R⁹, R¹¹ are independently of each other H or D, R¹⁰ is selected from H, D, NH₂, —CH₃, —CH₂D, —CHD₂ and —CD₃, particularly from H, D and NH₂, R¹² to R²² are independently of each other H or D.

To reduce the risk of loss of deuterium, the hydroxy ester moiety may be fully deuterated.

In certain embodiments, R⁸ is selected from D and —CD₃.

In certain embodiments, R⁹, R¹¹ are D.

In certain embodiments, R¹⁰ is selected from D, NH₂, and —CD₃, particularly from D and NH₂.

In certain embodiments, R⁸ is selected from D, and —CD₃, R⁹, R¹¹ are D, R¹⁰ is selected from D, NH₂, and —CD₃, particularly from D and NH₂.

In certain embodiments, R¹², R¹³, R¹⁴, R¹⁵, R¹⁶ are D.

In certain embodiments, R¹⁷, R¹⁸, R¹⁹, R²⁰, R²¹, R²² are D.

In certain embodiments, R⁸ is selected from D, and —CD₃, R⁹, R¹¹ are D, R¹⁰ is selected from D, NH₂, —CD₃, particularly from D and NH₂, R¹² to R²² are D.

In certain embodiments, Re is H or —OCH₃, and R⁷ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with R^{a} being H or a C₁₋₃-alkyl, and p being an integer between 0 and 6.

In case of Q being N, the protecting group Z may be selected from an amine protection group.

In certain embodiments, Q is N and Z is selected from Boc, Fmoc, benzyl carbamate, acetamide, trifluoroacetamide.

In case of Q being O, the protecting group Z may be a photolabile protecting group such as nitrobenzoyl.

In certain embodiments, Q is O and Z is selected from nitrobenzoyl and the following moieties:

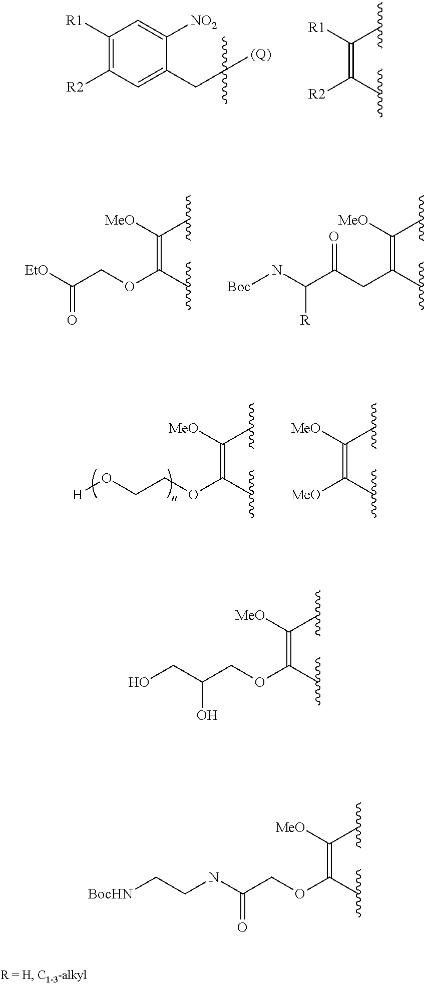

Prior to performing the method as described above, deuteration may be performed to achieve higher signal enhancement. Late-stage deuteration is not feasible as the required amount of base would lead to ester hydrolysis.

In certain embodiments, Z is a photolabile protecting group selected from

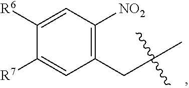

wherein
R⁶ or R⁷ is H or —OCH₃, and the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with R^{a} being H or a C₁₋₃-alkyl, p being an integer between 0 and 6.

Furthermore, using ¹³C labelled compounds of formula (VIIa), (VIIb) or (VIIc), particularly (VIIa), helps to further increase signals compared to unlabeled compounds.

The vinylation is performed in the presence of a catalyst for transfer esterification such as a Pd(0) and/or Pd(2+) catalyst.

In certain embodiments, the vinylation in step (a) is performed using Pd(OAc)₂.

For deuterated compounds, it showed using freshly recrystallized Pd(OAC)₂ increases the yield by 10%.

In certain embodiments, the vinylation step (a) is performed using freshly recrystallized Pd(OAC)₂.

In certain embodiments, the vinylation step (a) yields an intermediate of formula (VIIIa), (VIIIb) or (VIIIc), wherein each of E¹, E², E³, E⁴ and E⁵ is

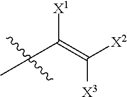

with X¹, X² and X³ as defined herein.

To avoid the loss of deuterium at the vinyl moiety, reaction conditions have to be carefully controlled.

In case of Q being-O—, a photolabile protecting group Z as described above may be used. Removal of the protecting group is achieved by applying UV light.

In certain embodiments, the UV light in step (b) has a wave length between 200 nm and 500 nm, particularly 320 nm.

In case of Q being —N—, an amine protecting group Z as described above may be used. Removal of the amine protecting group may be performed by using suitable agents known to those of skill in the art. For example, the Boc protecting group may be removed by using TFA in a non-protic solvent.

In certain embodiments, Z is Boc.

In certain embodiments, removal of the protecting group in step (b) is performed by using TFA, particularly TFA in a non-protic solvent.

For a successful functional group inversion, it is important to remove excess acid to enhance yields. Therefore, co-evaporation of TFA with methanol and pH control are needed.

In certain embodiments, TFA is removed by co-evaporation with methanol in step (c).

The method according to claim 10, wherein the TFA is removed in step (c) until the molar amount of TFA in relation to the molar amount of the compound of formula (VIIIa), (VIIIb) or (VIIIc) is 0 to 1, particularly 0 to 0.2, more particularly 0 to 0.1.

If the vinylation in step (a) was performed in such a way that maximum vinylation was achieved, the vinyl hydroxy esters obtained in step (d) are also fully vinylated.

In certain embodiments, each of E¹, E², E³, E⁴ and E⁵ of the vinyl hydroxy ester of formula (IXa), (IXb) or (IXc) is

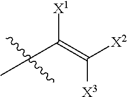

with X¹, X² and X³ as defined herein.

Addition of sodium nitrite as pure salt as well as solution in one portion in step (d) led to decomposition.

In certain embodiments, the addition of nitrite in step (d) is performed in small portions, particularly dropwise.

In certain embodiments, the bromide, particularly NaBr, for the bromination in step (d) is added before the addition of nitrite, wherein particularly the addition of nitrite is performed in small portions, particularly dropwise.

In contrast to the acid, the TFA anion anion was found to be important for the vinyl ester stability. Synthetic procedures using chlorides or sulfates were proven to be unsuccessful.

In certain embodiments, converting Q into an alcohol in step (d) is performed in the presence of nitrite, particularly NaNO₂ or tert-butyl-NO₂, and an anion, particularly TFA anion.

In certain embodiments, converting Q into an alcohol in step (d) is performed at pH 5.5 to pH 7.

In certain embodiments, converting Q into bromine in step (d) is performed in the presence of nitrite, particularly NaNO₂ or tert-butyl-NO₂, more particularly NaNO₂, and an anion, particularly TFA anion.

In certain embodiments, the hydrolysis after converting Q into bromine in step (d) is performed at pH 8 to pH 9. The pH may be adjusted by using K₂CO₃.

To avoid a loss of deuterium for example at the vinyl moiety, the reactions in step (d) may be performed in D₂O. In this case, a deuterated alcohol moiety is introduced.

In certain embodiments, R′ is D.

For the preparation of a contrast agent for signal enhanced magnetic resonance imaging, a carbon of the vinyl hydroxy ester is hyperpolarized through ¹H-polarization transfer via pH₂ by standard methods The ester obtained may be used as such as contrast agent or may be cleaved by hydrolysis to use the hyperpolarized metabolite such as lactate as contrast agent.

In certain embodiments, the vinyl hydroxy ester of formula (IXa), (IXb) or (IXc) is hyperpolarized after step (d), or the vinyl hydroxy ester of formula (IXa), (IXb) or (IXc) hyperpolarized and hydrolysed after step (d).

In certain embodiments, the vinyl hydroxy ester of formula (IXa), (IXb) or (IXc) is hyperpolarized after step (d) yielding a hyperpolarized vinyl hydroxy ester.

In certain embodiments, the hyperpolarized vinyl hydroxy ester is hydrolysed.

In certain embodiments, at least one C atom of the compound of formulas (IXa), (IXb) and (IXc) is ¹³C.

In certain embodiments, the method steps described above are performed at a temperature between 15° C. and 35° C., particularly between 20° C. and 25° C.

A second aspect of the invention relates to a vinyl hydroxy ester of formula (IXa), (IXb) or (IXc), particularly (IXa),

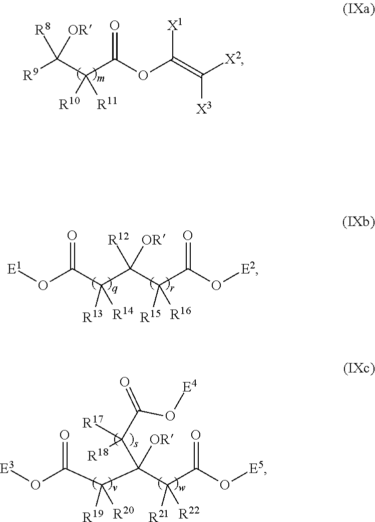

wherein
each R′ is independently of any other R′ selected from H and D, particularly H,
R⁸, R⁹ and R¹² are independently of each other selected from H, D, and a fully or partly deuterated alkyl, particularly C₁₋₁₆-alkyl, more particularly C₁₋₆-alkyl, even more particularly C₁₋₃-alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group, particularly selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
X¹, X² and X³ are independently of each other H or D, wherein at least one X¹, X² and X³ is D, particularly each of X¹, X² and X³ is D,
A is —CH₃, —CH₂D, —CHD₂ or —CD₃, particularly —CD₃, and
E¹, E², E³, E⁴ and E⁵ are independently of each other H, D or

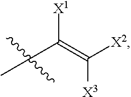

wherein X¹, X² and X³ are as defined above and wherein at least one moiety selected from E¹ and E² and at least one moiety selected from E³, E⁴ and E⁵ is

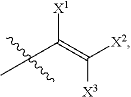

m, q, r, s, v and w are independently of each other integers between 0 and 16, particularly between 0 and 6, more particularly between 0 and 3.

In certain embodiments, at least one C atom of the compound of formulas (IXa), (IXb) and (IXc) is ¹³C.

In certain embodiment, the compound of formula (IXa) is partly or fully deuterated vinyl lactate or vinyl hydroxybutyrate, the compound of formula (IXb) is partly or fully deuterated vinyl malate and the compound of formula (IXc) is partly or fully deuterated vinyl citrate.

In certain embodiments, the compound of formula (IXa) is partly or fully deuterated vinyl lactate or vinyl hydroxybutyrate. In certain embodiments, the compound of formula (IXb) is partly or fully deuterated vinyl malate. In certain embodiments, the compound of formula (IXc) is partly or fully deuterated vinyl citrate.

Reference is made to the embodiments of the first aspect of the invention, particularly with regard to the definitions of R′, R⁸, R⁹, R¹⁰, R¹¹, R¹², R¹³, R¹⁴, R¹⁵, R¹⁶, R¹⁷, R¹⁸, R¹⁹, R²⁰, R²¹, R²², X¹, X², X³, E¹, E², E³, E⁴, E⁵, m, q, r, s, v and w.

A third aspect of the invention relates to an intermediate of formula (VIIIa), (VIIIb) or (VIIIc), particularly (VIIIa),

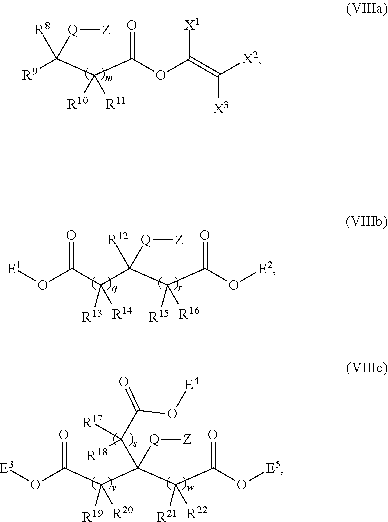

wherein
R⁸, R⁹ and R¹² are independently of each other selected from H, D, and a fully or partly deuterated alkyl, particularly C₁₋₁₆-alkyl, more particularly C₁₋₆-alkyl, even more particularly C₁₋₃-alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group, particularly selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
Q is O or N,
Z is a protecting group, particularly selected from, Boc, Fmoc, benzyl carbamate, acetamide, trifluoroacetamide, and

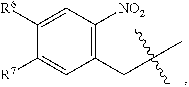

wherein
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
   R^{a} being H or a C₁₋₃-alkyl,
   p being an integer between 0 and 6,
X¹, X² and X³ are independently of each other H or D, wherein at least one X¹, X² and X³ is D, particularly each of X¹, X² and X³ is D,
A is —CH₃, —CH₂D, —CHD₂ or —CD₃, particularly —CD₃, and
E¹, E², E³, E⁴ and E⁵ are independently of each other H, D or

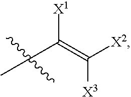

wherein X¹, X² and X³ are as defined above and wherein at least one moiety selected from E¹ and E² and at least one moiety selected from E³, E⁴ and E⁵ is

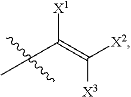

m, q, r, s, v and w are independently of each other integers between 0 and 16, particularly between 0 and 6, more particularly between 0 and 3.

In certain embodiments the intermediate of formula (VIIIa), (VIIIb) or (VIIIc), particularly (VIIIa),

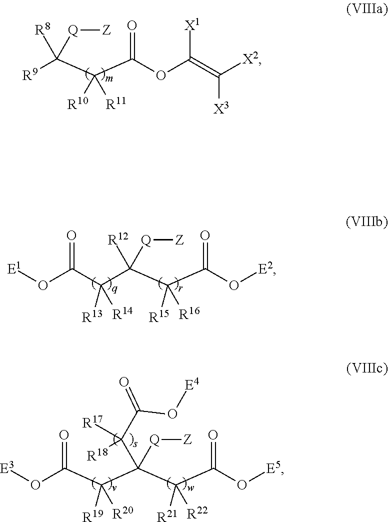

is characterized by
R⁸, Ro and R¹² are independently of each other selected from H, D, and a fully or partly deuterated alkyl, particularly C₁₋₁₆-alkyl, more particularly C₁₋₆-alkyl, even more particularly C₁₋₃-alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group, particularly selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
Q is O,
Z is a photolabile protecting group, particularly selected from

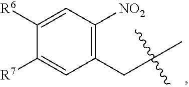

wherein
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
   R^{a} being H or a C₁₋₃-alkyl,
   p being an integer between 0 and 6,
X¹, X² and X³ are independently of each other H or D, wherein at least one X¹, X² and X³ is D, particularly each of X¹, X² and X³ is D,
A is —CH₃, —CH₂D, —CHD₂ or —CD₃, particularly —CD₃, and
E¹, E², E³, E⁴ and E⁵ are independently of each other H, D or

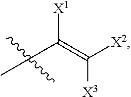

wherein X¹, X² and X³ are as defined above and wherein at least one moiety selected from E¹ and E² and at least one moiety selected from E³, E⁴ and E⁵ is

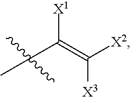

m, q, r, s, v and w are independently of each other integers between 0 and 16, particularly between 0 and 6, more particularly between 0 and 3.

In certain embodiments, at least one C atom of the moieties

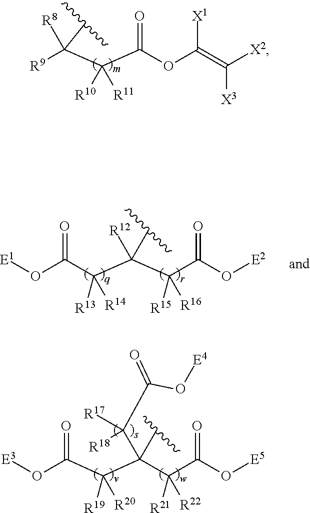

of the intermediates of formula (VIIIa), (VIIIb) or (VIIIc) is ¹³C.

Reference is made to the embodiments of the first aspect of the invention, particularly with regard to the definitions of R′, R⁸, R⁹, R¹⁰, R¹¹, R¹², R¹³, R¹⁴, R¹⁵, R¹⁶, R¹⁷, R¹⁸, R¹⁹, R²⁰, R²¹, R²², X¹, X², X³, E¹, E², E³, E⁴, E⁵, Q, Z, m, q, r, s, v and w.

In certain embodiments of any aspect of the present invention, one or more protons are replaced by deuterium. The molecules described herein may be fully deuterated.

The invention is further illustrated by the following examples, from which further embodiments and advantages can be drawn. These examples are meant to illustrate the invention but not to limit its scope.

### EXAMPLES

### Example 1: Synthesis of Vinyl Hydroxy Esters

Deuterated vinyl lactate (5) was synthesized via two different synthesis routes (B, C) by using nitrite as shown in Scheme 3 and described below. Subsequently, vinyl lactate 5 was hyperpolarized and cleaved (D) to obtain an NMR contrast agent (6).

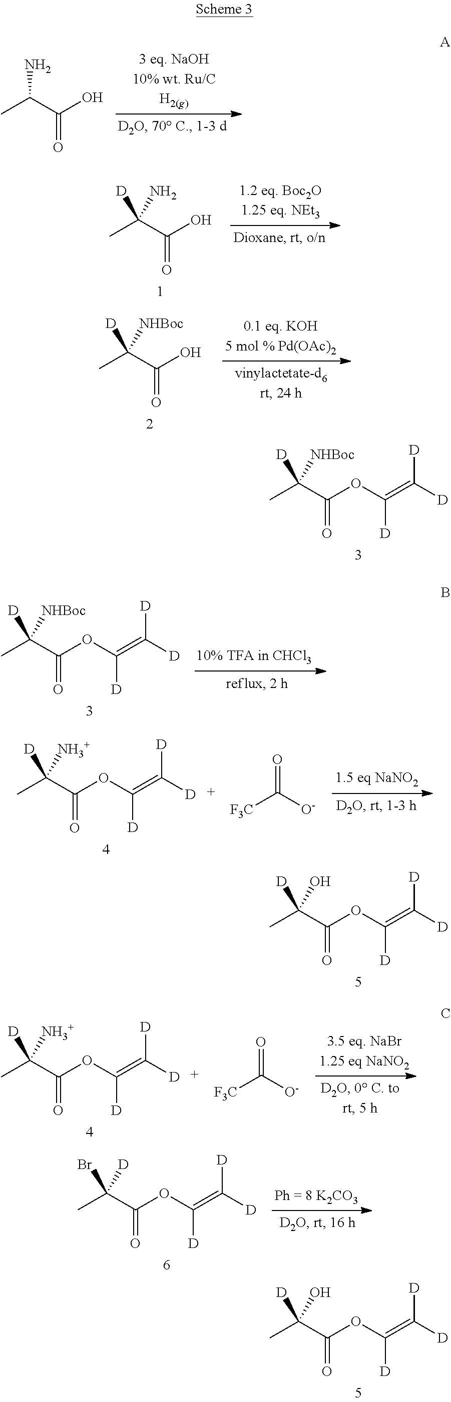

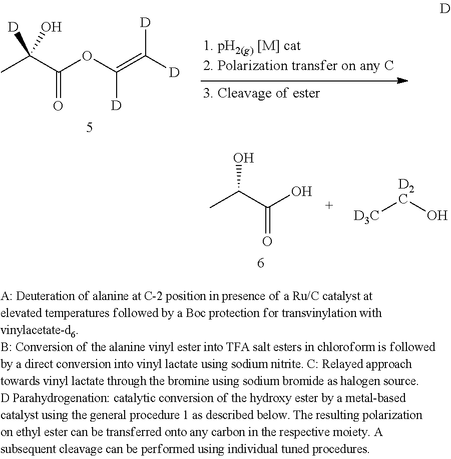

Panel C shows an alternative route for the synthesis of compound 5 starting from compound 4 as compared to the respective synthesis steps shown in panel B.

### Synthesis of Compound 1 (Panel A in Scheme 3)

This step is optional. Deuteration of C-2 however can lead to higher signal enhancements of 2.

Using ¹³C labeled alanine helps to further increase signals compared to unlabeled compounds. Late-stage deuteration is not feasible as the required amount of base would lead to ester hydrolysis. Nevertheless, the following reactions work on both, protonated and deuterated products.

A three-neck round bottom flask was charged with L-Ala (8.9 g, 99.9 mmol, 1 eq.), ruthenium on carbon (0.89 g, 10% wt) and sodium hydroxide (12 g, 299.7 mmol, 3 eq.). The sealed flask was filled with inert gas and D₂O was added. The atmosphere was changed from N₂ to H₂ by evacuation. To activate the catalyst, H₂ has to be bubbled through the reaction media. The reaction was warmed to 70° C. for 1-3 d. Reaction control is performed by NMR. After full conversion, the reaction was cooled to room temperature, filtered through Celite and the pH was decreased to 6. Then Dowex X-8 resin was added and washed with 25 mL aqueous solution of ammonia. The solvent was evaporated under reduced pressure. The L-d-2-Alanine is found as a yellow solid (98% deuteration). (Michelotti et al. 2017)

### Synthesis of Compound 2 (Panel A in Scheme 3)

The amino acid 1 (4.5 g, 49.94 mmol, 1 eq.) was dissolved in water at 0° C. before NEt₃ (7.58 g, 74.92 mmol, 1.25 eq.) was added. Boc₂O (13.08 g, 59.93 mmol, 1.2 eq.) was dissolved in dioxane (40 mL) and added to the reaction mixture. The solution was stirred at room temperature overnight. The solvent was removed in vacuo and the resulting residue was dissolved in ethyl acetate and extracted D₂O for 3 times. The organic phase was dried and concentrated under reduced pressure.

### Synthesis of Compound 3 (Panel A in Scheme 3)

For deuterated compounds, it showed using freshly recrystallized Pd(OAC)₂ increases the yield by 10%.

The Boc-protected-2D-alanine 2 (0.25 g, 1.31 mmol, 1 eq.) was dissolved in vinyl acetate-d₆ (0,716 g, 7.86 mmol, 6 eq.) and stirred until the solution turns clear. To the stirring solution palladium acetate (0.003 g, 0.01 mmol, 0.01 eq.) and potassium hydroxide (0.007 g, 0.13 mmol, 0.1 eq.) were added. The reaction mixture was stirred at room temperature for 24 h. The remaining vinyl acetate was removed in vacuo. The crude reaction mixture was loaded on a silica gel column (PE:EtOAc, 10:1, Rf: 0.3). —NHBoc-alanine-vinyl-d3 ester 3 (0.21 g, 0.98 mmol, 75%) was obtained as a crystalline white powder. (Saikiran et al. 2017)

### Synthesis of Compound 4 (Panel B in Scheme 3)

For a successful functional group inversion, it is important to remove excess acid to enhance yields. Therefore, co-evaporation of TFA with methanol and pH control are needed.

A flame-dried flask was charged with the Boc-N-Ala-vinyl ester 3 (0.2 g, 0.92 mmol, 1 eq.). Dichloromethane (8 mL) and trifluoroacetic acid (0.8 mL) were added and the reaction mixture was stirred for 3 hours at room temperature. After this time, 5 mL methanol were added to the crude reaction mixture and the solvent was evaporated in vacuo. The addition of methanol with subsequent evaporation was repeated until a steady weight of the round bottom flask was reached. A small sample was taken for analytical purposes. The product was used for the next reaction without any further purification (0.211 g, 0.92 mmol, 99%). (Kaltschnee et al. 2019)

### Synthesis of Compound 5 (Panel B in Scheme 3)

Synthetic procedures using chlorides or sulfates were proven to be unsuccessful. The presence of the TFA anion was found to be essential for the vinyl ester stability.

The amino salt vinyl ester 4 (0.211 g, 0.93 mmol, 1 eq.) was dissolved in D₂O (9 mL, concentrations of salt from 70-100 mM, pH range from 5.5 to 7.5). A sodium nitrite solution (0.18 g, 2.60 mmol, 1.5 eq. in 10% of total reaction volume) was added in small portions over 30 min to the stirring solution. Addition of sodium nitrite as pure salt as well as solution in one portion led to decomposition. The reaction was stirred for 1-3 h until the gas evolution stopped. The volume of the reaction was doubled by adding more D₂O followed by the extraction of the alcohol using diethyl ether. After combining the organic fractions, the crude solution was concentrated under reduced pressure. The crude alcohol was purified by silica column chromatography (PE:EtOAc, 4:1, Rf: 0.28). Vinyl lactate 5 (0.70 g, 0.56 mmol, 61%) was obtained as a yellow oil.

### Synthesis of Compound 6 (Panel C in Scheme 3)

Alanine vinyl ester trifluoroacetate salt 4 (0.97 g, 4.27 mmol, 1 eq.) was dissolved in D₂O (25 mL). The stirring solution was cooled to 0° C. and sodium bromide (1.62 g, 15.78 mmol, 3.5 eq.) was added and stirred for 20 minutes. The sodium bromide has to be added before the addition of sodium nitrite, else yields are significantly lower. After this time sodium nitrite (0.47 g, 6.75 mmol, 1.25 eq.) was added in small portions. After one hour the reaction was warmed to room temperature and stirred for additional 5 h. The aqueous phase was extracted with EtOAc (5×10 mL). The combined organic layers were combined and concentrated under reduced pressure by rotatory evaporation. The crude product was purified by column chromatography (PE:EtOAc, 8:1 Rf: 0.29). Vinyl-bromopropionate 6 (0.55 g, 3.10 mmol, 72%) was obtained as a yellow oil.

### Synthesis of Compound 5 (Panel C in Scheme 3)

Vinyl-bromopropionate 6 (0.1 g, 0.56 mmol) was dissolved in a pH 8 solution of K₂CO₃ in D₂O (10 mL). The reaction was stirred for 16 h. After this time, the aqueous phase was extracted with EtOAc (10×5 mL). The combined organic fractions were combined and concentrated under reduced pressure by rotatory evaporation. The crude alcohol was purified by silica column chromatography (PE:EtOAc, 4:1, Rf: 0.28). Vinyl lactate 5 (0.05 g, 0.46 mmol, 83%) was obtained as a yellow oil.

### General Procedure 1: Para Hydrogenation of Unsaturated Bonds (Panel D in Scheme 3)

Para hydrogenation may be performed by methods known to those of skill in the art.

A stock solution 1 of the unsaturated molecules was made by dissolving the compound in an appropriate solvent (D₂O, CDCl₃, CO(CD₃)₂ and comparable) with a final concentration of 2 to 10 M. The compatible stock solution 2 of the metal-based catalyst was made by dissolving the compound in the same solvent with concentrations of 1-4 mM or 2-5 g/mL. A total volume of 400 μL were composed of stock solution 1 (final concentrations of 0.5-3 mM), stock solution 2 (0.5-3 mM or 0.1-2 g/mL) and the solvent (filling up to 400 μL) and transferred to a pressure safe NMR tube. The bubbling setup is attached, the tube is placed in the pre-warmed (T=353 K) spectrometer and the solution was allowed to warm to the required temperatures. Then, the optimized pulse sequence (e.g. ESOTHERIC; with adjusted bubbling and settle time which depends on the respective unsaturated compound) is executed.

### Example 2: Synthesis of Vinyl Hydroxy Esters Using a Photolabile Protection Group

Scheme 4 shows the synthesis of vinyl lactate using nitrobenzoyl as protecting group. The vinyl moiety may be deuterated.

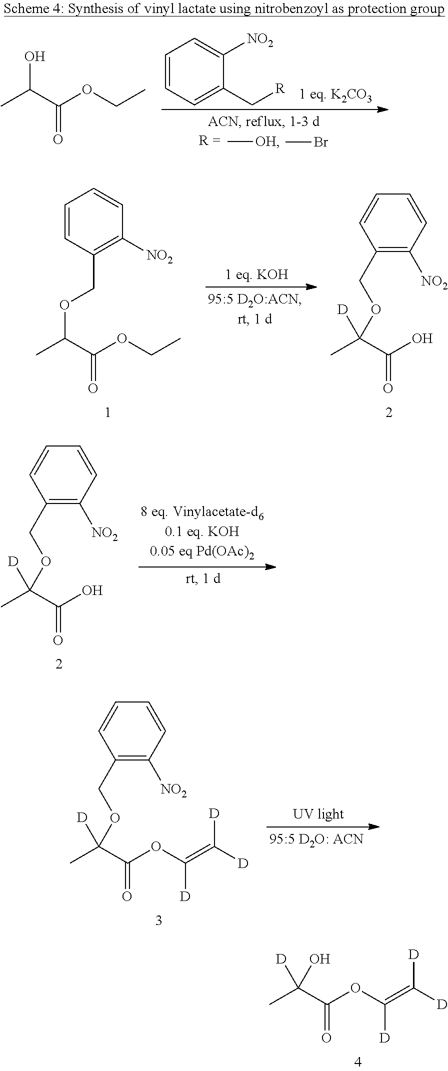

### Synthesis of 1: ethyl 2-((2-nitrobenzyl)oxy)propanoate

In a flame dryed flask under innert atmosphere ethyl lactate (0.169 g, 1,429 mmol, 1 eq.), 1-(bromomethyl)-2-nitrobenzene (0.309 g, 1.429 mmol, 1 eq.) or 1-(methanol)-2-nitrobenzene (0.219 g, 1.43 mmol, 1 eq.) and K₂CO₃ (0.198 g, 1.43 mmol, 1 eq.) were dissolved in dry acetonitrile (5.7 mL). The mixture was heated to reflux for 1 to 3 days. The reaction was controlled by TLC (PE:EtOAc 5:1, Rf 0.25). The reaction was allowed to cool to rt before adding X mL H₂O. The aqueous layer was extracted with EtOAc (3×10 mL) and the combined organic layers were concentrated under reduced pressure. The crude product was purified by flash column chromatography using a gradient solvent system (from 95:5 PE:EtOAc to 60:40 PE:EtOAc). The pure ether was collected as a dark brown oil.

### Synthesis of 2: 2-((2-nitrobenzyl)oxy)propanoic-2-d Acid

The ester 1 (0.15 g, mmol, eq.) was dissolved in 10 mL D₂O containing 5% acetonitrile-d₃ and mixed for 10 min. KOH (0,022 g, 0,395 mmol, 1 eq.) was added in one portion, the flask was sealed and stirred for 1 d. After this time, CH₂Cl₂ (3×5 mL) was used to extract the free acid. Concentration of the combined organic layers gave the pure acid as a brown solid.

### Synthesis of 3: vinyl-d₃-2-((2-nitrobenzyl)oxy)propanoate-2-d

The 2-Nitrobenzyl protected acid 2 (0.25 g, 1.11 mmol, 1 eq.) was dissolved in vinyl acetate-d₆ (3 mL) and stirred until the solution turns clear. To the stirring solution palladium acetate (0.012 g, 0.056 mmol, 0.05 eq.) and potassium hydroxide (0.06 g, 0.111 mmol, 0.1 eq.) were added. The reaction mixture was stirred at room temperature for 24 h. The remaining vinyl acetate was removed in vacuo. The crude reaction mixture was loaded on a silica gel column). Vinyl-d₃ ester 3 was obtained as a crystalline brown powder.

### Synthesis of 4: vinyl-d₃-lactate-2-d

The 2-Nitrobenzyl protected alcohol 3 (0.25 g, 0.995 mmol, 1 eq.) was dissolved in D₂O containing 5% acetonitrile-d₃ (total volume 12 mL) and irradiated for 30-60 min in a light reactor tuned for λ=365 nm light irradiation. Typically, concentrations of 50-100 mM, more precisely 80 mM were used to reduce side reactions due to induced radicals by the UV light. The aqueous solution was extracted with EtOAc (3×3 mL) and the combined organic layers were concentrated under reduced pressure. The crude vinyl-d₃-lactate-2-d 4 was purified by silica column chromatography.

### REFERENCES

Kaltschnee et al. (2019) “Hyperpolarization of Amino Acids in Water Utilizing Parahydrogen on a Rhodium Nanocatalyst”. Chemistry. A European Journal 25 (47): 11031-11035
Michelotti et al. (2017) “Development and Scale-Up of Stereoretentive α-Deuteration of Amines”. Org. Process Res. Dev. 21 (11): 1741-1744
Saikiran et al. (2017) “Efficient near infrared fluorescence detection of elastase enzyme using peptide-bound unsymmetrical squaraine dye”. Bioorganic & Medicinal Chemistry Letters 27 (17): 4024-4029

1. A method for preparing a compound suitable for signal enhanced magnetic resonance imaging comprising the steps of
a) vinylation of a compound of formula (VIIa), (VIIb) or (VIIc), 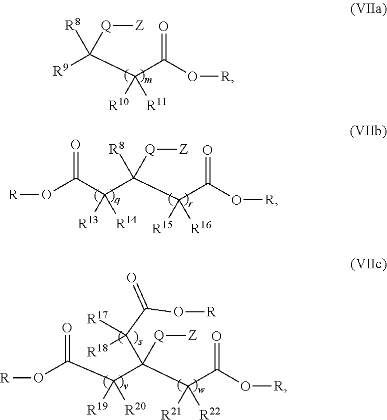
by using vinyl acetate of formula (II), 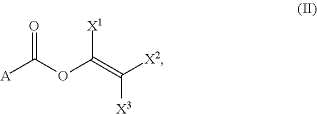
yielding an intermediate of formula (VIIIa), (VIIIb) or (VIIIc) 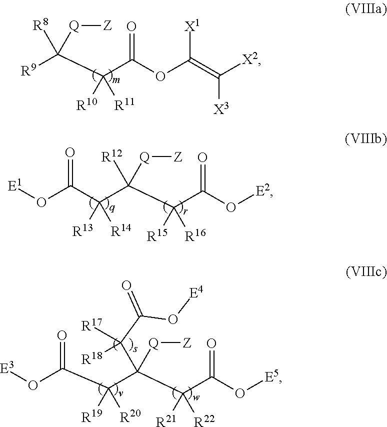
b) cleaving Z by using UV light or by using a suitable agent, particularly TFA,
c) in case of using TFA in step (b), TFA is removed,
d) converting Q into an alcohol in the presence of nitrite, particularly NaNO₂ or tert-butyl-NO₂, or
converting Q into bromine in the presence of nitrite, particularly NaNO₂ or tert-butyl-NO₂, followed by hydrolysis
yielding a vinyl hydroxy ester of formula (IXa), (LXb) or (IXc), 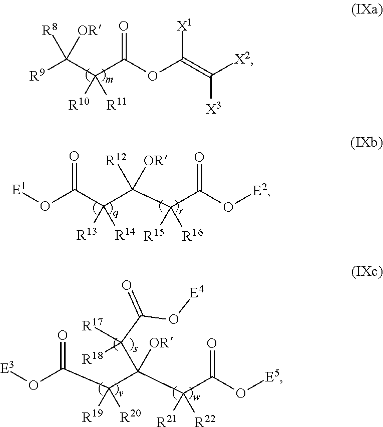
wherein
each R is independently of any other R selected from H and D, particularly H,
R′ is selected from H and D, particularly H,
R⁸, R⁹ and R¹² are independently of each other selected from H, D, and a fully or partly deuterated alkyl, particularly C₁₋₁₆-alkyl, more particularly C₁₋₆-alkyl, even more particularly C₁₋₃-alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
Q is O or N,
Z is a protecting group, particularly selected from an amine protection group and a photolabile protection group,
X¹, X² and X³ are independently of each other H or D,
A is —CH₃, —CH₂D, —CHD₂ or —CD₃, particularly —CD₃,
E¹, E², E³, E⁴ and E⁵ are independently of each other H, D or 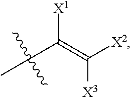
wherein X¹, X² and X³ are as defined above and wherein at least one moiety selected from E¹ and E² and at least one moiety selected from E³, E⁴ and E⁵ is 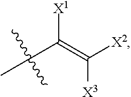
and
m, q, r, s, v and w are independently of each other integers between 0 and 16, particularly between 0 and 6, more particularly between 0 and 3.

2. The method according to claim 2, wherein X¹, X² and X³ are D.

3. The method according to claim 1, wherein at least one C atom of the compound of formulas (IXa), (IXb) and (IXc) is ¹³C.

4. The method according to claim 1, wherein Z is a protecting group selected from, Boc, Fmoc, benzyl carbamate, acetamide, trifluoroacetamide, and 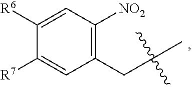
wherein
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
R^{a} being H or a C₁₋₃-alkyl,
p being an integer between 0 and 6.

5. The method according to claim 1, wherein R⁶ is H or —OCH₃, and R⁷ is selected from H, —OCH³, —O—CH₂—C(═O)—O—CH₂—CH₃, —CH₂—C(═O)—CH(Rₐ)—NH-Boc, —O—[CH₂—CH₂—O]ₚ—H, —O—CH₂—CH(OH)—CH₂—OH, —O—CH₂—C(═O)—NH—CH₂—CH₂—NH-Boc, with R^{a} being H or a C₁₋₃-alkyl, and p being an integer between 0 and 6.

6. The method according to claim 1, wherein the TFA is removed in step (c) until the molar amount of TFA in relation to the molar amount of the compound of formula (VIIIa), (VIIIb) or (VIIIc) is 0 to 1, particularly 0 to 0.2, more particularly 0 to 0.1.

7. The method according to claim 1, wherein hydrolysing Q in step (d) is performed at pH 5.5 to pH 7.

8. The method according to claim 1, wherein the hydrolysis after brominating Q in step (d) is performed at pH 8 to pH 9.

9. The method according to claim 1, wherein the vinyl hydroxy ester of formula (IXa), (IXb) or (IXc) is hyperpolarized after step (d) yielding a hyperpolarized vinyl hydroxy ester.

10. The method according to claim 9, wherein the hyperpolarized vinyl hydroxy ester is hydrolysed.

11. The method according to claim 1, wherein the addition of nitrite in step (d) is performed in small portions, particularly dropwise.

12. A a vinyl hydroxy ester of formula (IXa), (IXb) or (IXc), 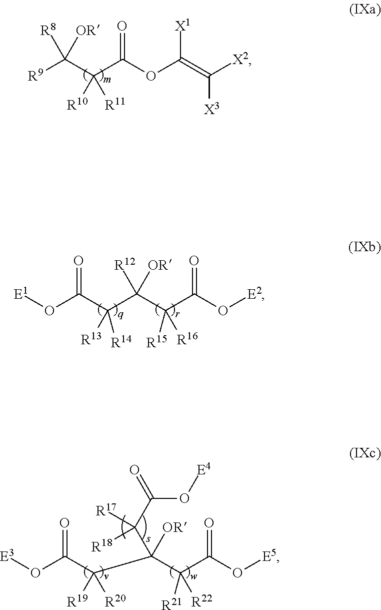
wherein
R′ is selected from H and D, particularly H,
R⁸, R⁹ and R¹² are independently of each other selected from H, D, and a fully or partly deuterated alkyl, particularly C₁₋₁₆-alkyl, more particularly C₁₋₆-alkyl, even more particularly C₁₋₃-alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group, particularly selected from H, D and NH₂,
each R¹¹, R¹⁴, R¹⁶, R¹⁸, R²⁰ and R²² is independently of any other R¹¹, R¹⁴, R¹⁶, R¹⁸, R²⁰ or R²² selected from H and D,
X¹, X² and X³ are independently of each other H or D, wherein at least one X¹, X² and X³ is D, particularly each of X¹, X² and X³ is D,
A is —CH₃, —CH₂D, —CHD₂ or —CD₃, particularly —CD₃, and
E¹, E², E³, E⁴ and E⁵ are independently of each other H, D or 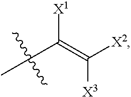
wherein X¹, X² and X³ are as defined above and wherein at least one moiety selected from E¹ and E² and at least one moiety selected from E³, E⁴ and E⁵ is 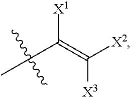
m, q, r, s, v and w are independently of each other integers between 0 and 16, particularly between 0 and 6, more particularly between 0 and 3.

13. The vinyl hydroxy ester according to claim 12, wherein at least one C atom of the compound of formulas (IXa), (IXb) and (IXc) is ¹³C.

14. An intermediate of formula (VIIIa), (VIIIb) or (VIIIc) 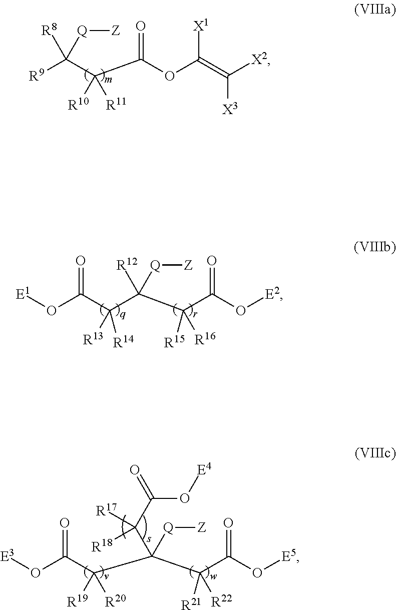
wherein
R⁸, R⁹ and R¹² are independently of each other selected from H, D, and a fully or partly deuterated alkyl, particularly C₁₋₁₆-alkyl, more particularly C₁₋₆-alkyl, even more particularly C₁₋₃-alkyl,
each R¹⁰, R¹³, R¹⁵, R¹⁷, R¹⁹ and R²¹ is independently of any other R¹⁰, R¹³, R¹⁵, R¹⁷, R¹⁹ or R²¹ selected from H, D and NH₂, wherein the NH₂ moiety may be protected by a protecting group, particularly selected from H, D and NH₂,
each R₁₁, R₁₄, R₁₆, R₁₈, R₂₀ and R₂₂ is independently of any other R₁₁, R₁₄, R₁₆, R¹⁸, R²⁰ or R²² selected from H and D,
Q is O or N,
Z is a protecting group, particularly selected from an amine protection group and a photolabile protection group, more particularly selected from, Boc, Fmoc, benzyl carbamate, acetamide, trifluoroacetamide, and 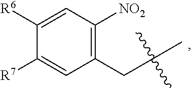
wherein
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
R^{a} being H or a C₁₋₃-alkyl,
p being an integer between 0 and 6,
X¹, X² and X³ are independently of each other H or D, wherein at least one X¹, X² and X³ is D, particularly each of X¹, X² and X³ is D,
A is —CH₃, —CH₂D, —CHD₂ or —CD₃, particularly —CD₃, and
E¹, E², E³, E⁴ and E⁵ are independently of each other H, D or 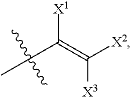
wherein X¹, X² and X³ are as defined above and wherein at least one moiety selected from E¹ and E² and at least one moiety selected from E³, E⁴ and E⁵ is 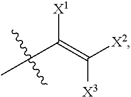
m, q, r, s, v and w are independently of each other integers between 0 and 16, particularly between 0 and 6, more particularly between 0 and 3.

15. The intermediate according to claim 14, wherein at least one C atom of the moieties 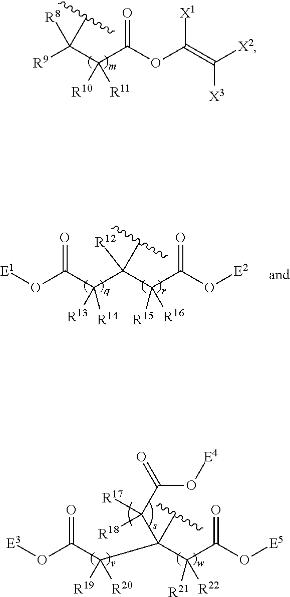
is ¹³C.
